(19) United States
(12) Patent Application Publication
(10) Pub. No.: US 2025/0057777 A1
(43) Pub. Date: Feb. 20, 2025

(54) Intestine-Targeted Drug Delivery Compositions and Preparation Methods Thereof

(71) Applicant: Hefei Cosource Pharmaceuticals Co., Ltd, Hefei (CN)

(72) Inventors: Yang Zhang, Hefei, Anhui Province (CN); Dengjun Chen, Hefei, Anhui Province (CN); Yuting Jin, Hefei, Anhui Province (CN); Qiongxia Xie, Hefei, Anhui Province (CN); Hongzhang Sun, Hefei, Anhui Province (CN)

(73) Assignee: Hefei Cosource Pharmaceuticals Co., Ltd, Hefei (CN)

(21) Appl. No.: 18/800,216

(22) Filed: Aug. 12, 2024

(30) Foreign Application Priority Data
Aug. 14, 2023 (CN) 202311029374.1

(51) Int. Cl. A61K 9/50; A61K 9/00; A61K 31/4985; A61K 31/519; A61K 31/573

(52) U.S. Cl. CPC A61K 9/5078; A61K 9/0053; A61K 9/501; A61K 9/5015; A61K 9/5042; A61K 9/5047; A61K 9/5073; A61K 31/4985; A61K 31/519; A61K 31/573

(57) ABSTRACT

The present application discloses an intestine-targeted drug delivery composition, comprising in sequence, from inside to outside, (1) a microsphere core and a pH-independent sustained-release layer containing an active ingredient, or a microsphere containing an active ingredient and a pH-independent sustained-release layer, or a microsphere containing an active ingredient and a pH-independent sustained-release material, (2) a pH-dependent coating layer, and (3) a time-dependent pulsatile controlled-release layer. The intestine-targeted drug delivery composition of the present application delivers the active ingredient to intestinal tissues (especially colon tissue) for release, reduces the systemic blood concentration of the active ingredient, and improves the pharmacokinetic characteristics and concentration of the active ingredient in intestinal tissues. The composition is suitable for treating intestinal diseases, such as inflammatory bowel diseases including Crohn's disease and ulcerative colitis.

### CROSS-REFERENCE TO RELATED APPLICATIONS

The present application claims the benefit and priority to the Chinese patent application No. 202311029374.1 filed before the China national intellectual property administration on Aug. 14, 2023, the content of which is incorporated herein by reference in its entirety.

### TECHNICAL FIELD

The present application belongs to the field of pharmaceutical formulations, specifically relates to intestine-targeted drug delivery compositions and preparation methods thereof, and more specifically relates to intestine-targeted drug delivery compositions containing JAK inhibitors, hormones, and/or other agents used for treating intestinal diseases, and preparation methods thereof.

### BACKGROUND

Inflammatory bowel diseases (IBD) are special chronic inflammatory bowel diseases, mainly including Crohn's disease (CD) and ulcerative colitis (UC).

JAK kinases belong to the cytoplasmic tyrosine kinase family and are believed to play an important role in inflammation. The inhibitors of the JAK kinase family have shown efficacy in treating inflammatory diseases (such as inflammatory bowel diseases including ulcerative colitis and Crohn's disease) and autoimmune diseases. Upadacitinib is apotent, selective and reversible JAK1 inhibitor, which is used for the treatment of moderate to severe rheumatoid arthritis, psoriatic arthritis, moderate to severe ulcerative colitis, ankylosing spondylitis, Crohn's disease, and other diseases. Tofacitinib is a JAK pathway inhibitor that has been approved for the treatment of rheumatoid arthritis and has good therapeutic effects in the treatment of various autoimmune diseases such as ankylosing spondylitis, psoriatic arthritis, inflammatory bowel diseases, systemic lupus erythematosus, and vitiligo.

However, the conventional formulations of JAK inhibitors for oral administration have been studied and found to have the following limitations: JAK inhibitors reach the intestinal sites, such as the colon site, in relatively low amounts, resulting in a reduced efficacy and poor patient compliance due to the need for long-term medication and significant adverse effects. Therefore, formulating JAK inhibitors as intestine-targeted delivery compositions becomes the best option.

Currently, the commonly used intestine-targeted drug delivery compositions mainly include pH-dependent intestine-targeted drug delivery compositions, time-dependent intestine-targeted drug delivery compositions, and gut microbiota/enzyme intestine-targeted drug delivery compositions, etc. However, the digestive environment and gastrointestinal tract transportation showed great variations in different populations. Gastric emptying is greatly influenced by the type of food and the time of eating, especially in patients with IBD due to mucosal lesions and dietary changes, which make the factors that determine the pH value of the colon change. It is difficult for the above intestine-targeted oral formulations to actually achieve precise colon-targeted release. Therefore, there is a need for new intestine-targeted drug delivery compositions to accurately deliver drugs to intestinal tissues, such as colon tissues, in order to achieve targeted release.

### SUMMARY

In one aspect, the present application provides an intestine-targeted drug delivery composition, which comprises in sequence, from inside to outside, (1) a microsphere core and a pH-independent sustained-release layer containing an active ingredient, or a microsphere containing an active ingredient and a pH-independent sustained-release layer, or a microsphere containing an active ingredient and a pH-independent sustained-release material, (2) a pH-dependent coating layer, and (3) a time-dependent pulsatile controlled-release layer.

In an another aspect, the present application provides a method for preparing an intestine-targeted drug delivery composition, comprising the steps of:

### (1) Preparation of a pH-Independent Sustained-Release Layer

Adding an active ingredient, a sustained-release coating film-forming material, and one or more pharmaceutically acceptable excipients selected from the group consisting of a plasticizer, a pore-forming agent, and an anti-sticking agent to an aqueous solution of an alcohol to form a sustained-release coating solution, and then coating blank microsphere cores with the sustained-release coating solution to obtain sustained-release microspheres; or

Adding a sustained-release coating film-forming material and one or more pharmaceutically acceptable excipients selected from the group consisting of a plasticizer, a pore-forming agent, and an anti-sticking agent to an aqueous solution of an alcohol to form a sustained-release coating solution; granulating an active ingredient, a binder, and a filler to obtain drug-containing microsphere cores; and then coating the drug-containing microsphere cores with the sustained-release coating solution to obtain sustained-release microspheres; or

Adding a sustained-release coating film-forming material and one or more pharmaceutically acceptable excipients selected from the group consisting of a plasticizer, a pore-forming agent, and an anti-sticking agent to an aqueous solution of an alcohol to form a sustained-release coating solution; coating blank microsphere cores with a solution containing an active ingredient and a binder to obtain drug-containing microsphere cores; and then coating the drug-containing microsphere cores with the sustained-release coating solution to obtain sustained-release microspheres; or

Granulating an active ingredient, a binder, a filler, and a pH-independent sustained-release material to obtain sustained-release microspheres containing the active ingredient and the pH-independent sustained-release material;

### (2) Preparation of a pH-Dependent Coating Layer

Adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the sustained-release microspheres obtained in step (1) with the pH-dependent coating solution to obtain pH-dependent microspheres; or

Adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the sustained-release microspheres obtained in step (1) with the pH-dependent coating solution to obtain pH-dependent microspheres; and mixing the pH-dependent microspheres with a filler, a disintegrant, and/or a lubricant, and then tableting to obtain a pH-dependent tablet; or

Mixing the sustained-release microspheres obtained in step (1) with a filler, a disintegrant, and/or a lubricant, and then tableting to obtain a compressed tablet; and adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the compressed tablet with the pH-dependent coating solution to obtain a pH-dependent tablet; and

### (3) Preparation of a Time-Dependent Pulsatile Controlled-Release Layer

Adding a permeation control material and a permeation regulating material to an aqueous solution of an alcohol to form a time-dependent coating solution, and then coating the pH-dependent microspheres or the pH-dependent tablet with the time-dependent coating solution to obtain the intestine-targeted drug delivery composition in microsphere form or tablet form.

In still another aspect, the present application provides a use of the intestine-targeted drug delivery composition mentioned above or the intestine-targeted drug delivery composition prepared by the method mentioned above in the manufacture of a medicament for treating intestinal diseases, such as inflammatory bowel diseases including Crohn's disease and ulcerative colitis.

In a further aspect, the present application provides a method for treating intestinal diseases, such as inflammatory bowel diseases including Crohn's disease and ulcerative colitis, comprising administering to a subject in need thereof the intestine-targeted drug delivery composition mentioned above or the intestine-targeted drug delivery composition prepared by the method mentioned above.

### BRIEF DESCRIPTION OF DRAWINGS

FIG. 1 shows a structural schematic diagram of an intestine-targeted drug delivery composition containing a drug according to the present application in the form of tablets. FIG. 1A shows a tablet formed from a tablet core excipient and drug-containing controlled-release microspheres and coated with a time-dependent pulsatile controlled-release layer. FIG. 1B shows a drug-containing controlled-release microsphere comprising in sequence, from inside to outside, a blank microsphere core, a drug-containing pH-independent sustained-release layer, and a pH-dependent coating layer. FIG. 1C shows a drug-containing controlled-release microsphere comprising in sequence, from inside to outside, a blank microsphere core, a drug layer, a pH-independent sustained-release layer, and a pH-dependent coating layer. FIG. 1D shows a drug-containing controlled-release microsphere comprising in sequence, from inside to outside, a drug-containing microsphere core, a pH-independent sustained-release layer, and a pH-dependent coating layer.

FIG. 2 shows another structural schematic diagram of an intestine-targeted drug delivery composition containing a drug according to the present application in the form of tablets. FIG. 2A shows a table formed from a tablet core excipient and drug-containing sustained-release microspheres and coated sequentially with a pH-dependent coating layer and a time-dependent pulsatile controlled-release layer. FIG. 2B shows a drug-containing sustained-release microsphere comprising in sequence, from inside to outside, a blank microsphere core and a drug-containing pH-independent sustained-release layer. FIG. 2C shows a drug-containing sustained-release microsphere comprising in sequence, from inside to outside, a blank microsphere core, a drug layer, and a pH-independent sustained-release layer. FIG. 2D shows a drug-containing sustained-release microsphere comprising in sequence, from inside to outside, a drug-containing microsphere core and a pH-independent sustained-release layer.

FIG. 3 shows dissolution curves of the intestine-targeted drug delivery compositions containing a drug prepared according to Comparative Examples 1 to 3 and Example 1.

FIG. 4 shows dissolution curves of the intestine-targeted drug delivery compositions containing a drug prepared according to Examples 1 to 4.

FIG. 5 shows drug plasma concentration-time curves in dogs of the intestine-targeted drug delivery compositions containing a drug prepared according to Comparative Example 1 and Comparative Example 2.

FIG. 6 shows drug plasma concentration-time curves in dogs of the intestine-targeted drug delivery compositions containing a drug prepared according to Comparative Example 3 and Example 1.

FIG. 7 shows drug concentration-time curves in the intestinal tissues of dogs of the intestine-targeted drug delivery compositions containing a drug prepared according to Comparative Example 1 and Comparative Example 2.

FIG. 8 shows drug concentration-time curves in the intestinal tissues of dogs of the intestine-targeted drug delivery compositions containing a drug prepared according to Comparative Example 3 and Example 1.

FIG. 9 shows drug plasma concentration-time curves in dogs of the intestine-targeted drug delivery compositions prepared according to Examples 1, 2, and 4 and the conventional tablet of tofacitinib citrate, after oral administration.

FIG. 10 shows drug concentration-time curves in the intestinal tissues of dogs of the intestine-targeted drug delivery composition prepared according to Example 1 and the conventional tablet of tofacitinib citrate, after oral administration.

### DETAILED DESCRIPTION

In one aspect, the present application provides an intestine-targeted drug delivery composition, which comprises in sequence, from inside to outside, a pH-independent sustained-release layer, a pH-dependent coating layer, and a time-dependent pulsatile controlled-release layer.

Specifically, the present application provides an intestine-targeted drug delivery composition, which comprises in sequence, from inside to outside, (1) a microsphere core and a pH-independent sustained-release layer containing an active ingredient, or a microsphere containing an active ingredient and a pH-independent sustained-release layer, or a microsphere containing an active ingredient and a pH-independent sustained-release material, (2) a pH-dependent coating layer, and (3) a time-dependent pulsatile controlled-release layer.

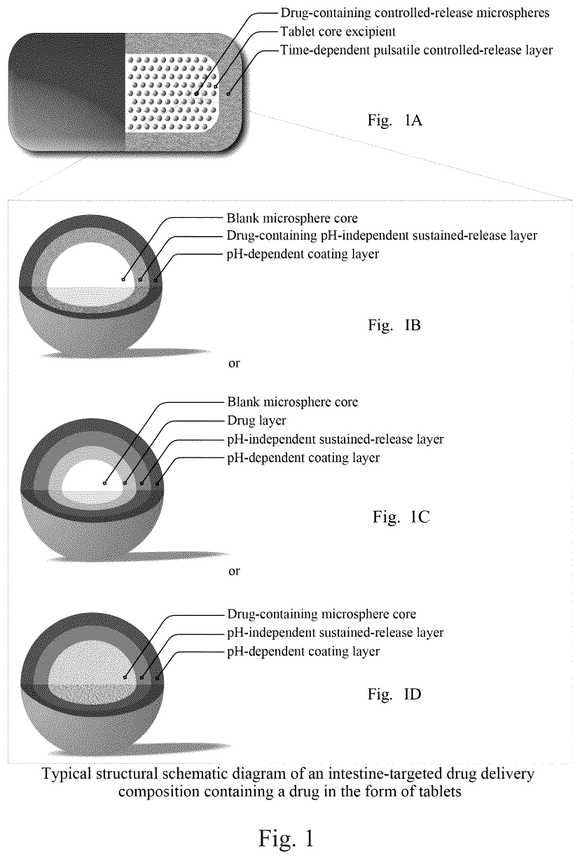

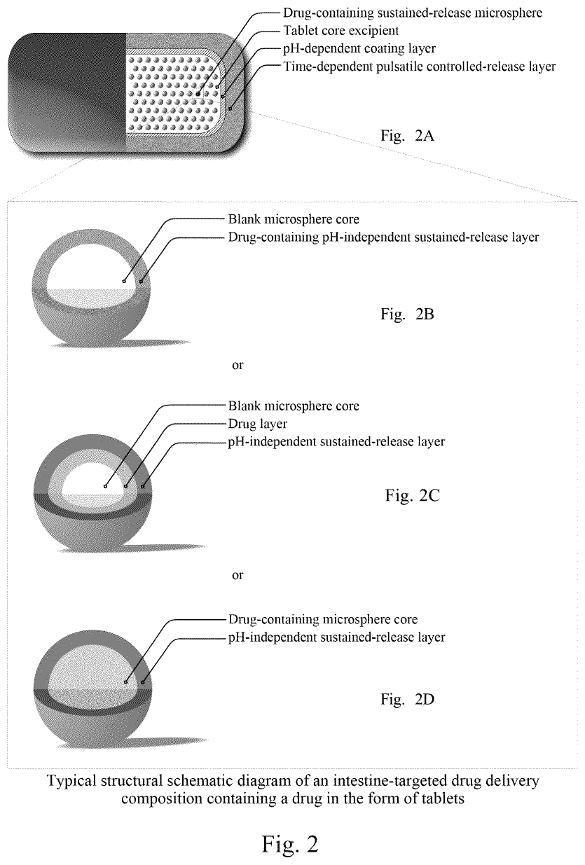

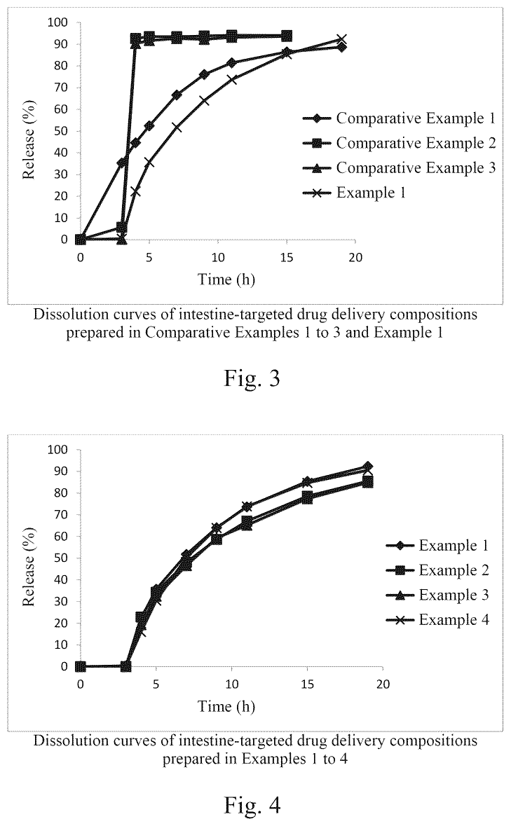

FIG. 1 and FIG. 2 respectively show two typical structural schematic diagrams of the intestine-targeted drug delivery compositions containg a drug in the form of a tablet according to the present application. In FIG. 1A, a tablet, which is formed by a tablet core excipient and drug-containing controlled-release microspheres comprising in sequence, from inside to outside, a pH-independent sustained-release layer and a pH-dependent coating layer, is coated with a time-dependent pulsatile controlled-release layer. In FIG. 2A, a tablet, which is formed by a tablet core excipient and drug-containing sustained-release microspheres comprising a pH-independent sustained-release layer, is coated sequentially with a pH-dependent coating layer and a time-dependent pulsatile controlled-release layer.

In some embodiments of the present application, the pH-independent sustained-release layer may be a sustained-release layer containing an active ingredient. For example, as shown in FIG. 1B and FIG. 2B, the active ingredient is present in the pH-independent sustained-release layer. That is, a sustained-release layer containing the active ingredient is provided on an outer surface of a blank microsphere core. Alternatively, the active ingredient is mixed with a pH-independent sustained-release material and other suitable excipients to form a mixture, which is then granulated to form microspheres containing the active ingredient and the pH-independent sustained-release material.

In some embodiments of the present application, the pH-independent sustained-release layer may be a sustained-release layer without an active ingredient. For example, as shown in FIG. 1C and FIG. 2C, the active ingredient is present between the sustained-release layer and the blank microsphere core. That is, an active ingredient layer and the sustained-release layer are sequentially provided on an outer surface of the blank microsphere core. Alternatively, as shown in FIG. 1D and FIG. 2D, the active ingredient is present within the microsphere core to form a drug-containing microsphere core, and the sustained-release layer is provided on the outer surface of the drug-containing microsphere core.

In addition, the intestine-targeted drug delivery compositions containing a drug according to the present application may be in the form of capsules or granules. The capsules or granules contain microspheres that comprise in sequence, from inside to outside, a pH-independent sustained-release layer, a pH-dependent coating layer, and a time-dependent pulsatile controlled-release layer, wherein the pH-independent sustained-release layer is a sustained-release layer with or without the active ingredient as described above.

In some embodiments of the present application, the pH-independent sustained-release layer comprises a sustained-release coating film-forming material and one or more pharmaceutically acceptable excipients selected from the group consisting of a plasticizer, a pore-forming agent, and an anti-sticking agent. In some embodiments of the present application, the pH-independent sustained-release layer comprises a sustained-release coating film-forming material, a plasticizer, and one or more pharmaceutically acceptable excipients selected from the group consisting of a pore-forming agent and an anti-sticking agent. In some embodiments of the present application, the pH-independent sustained-release layer comprises a sustained-release coating film-forming material, a pore-forming agent, and one or more pharmaceutically acceptable excipients selected from the group consisting of a plasticizer and an anti-sticking agent. In some embodiments of the present application, the pH-independent sustained-release layer comprises a sustained-release coating film-forming material, a plasticizer, and a pore-forming agent, and optionally an anti-sticking agent.

In some embodiments of the present application, the sustained-release coating film-forming material is selected from the group consisting of methacrylate copolymer, ethyl cellulose, hydroxypropyl methylcellulose, cellulose acetate, and combinations thereof. Alternatively, the sustained-release coating film-forming material is selected from the group consisting of ethyl cellulose, methacrylate copolymer, cellulose acetate, and combinations thereof. Alternatively, the sustained-release coating film-forming material is ethyl cellulose.

In some embodiments of the present application, the pH-independent sustained-release material is selected from the group consisting of methacrylate copolymer, ethyl cellulose, hydroxypropyl methylcellulose, cellulose acetate, and combinations thereof. Alternatively, the pH-independent sustained-release material is one or both of ethyl cellulose and hydroxypropyl methylcellulose.

In some embodiments of the present application, the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof. Alternatively, the plasticizer is selected from the group consisting of triethyl citrate and glycerol triacetate. Alternatively, the plasticizer is triethyl citrate.

In some embodiments of the present application, the pore-forming agent is selected from the group consisting of polyethylene glycol, hydroxypropyl methylcellulose, copovidone, povidone, and combinations thereof. Alternatively, the pore-forming agent is selected from the group consisting of polyethylene glycol and hydroxypropyl methylcellulose. Alternatively, the pore-forming agent is hydroxypropyl methylcellulose.

In some embodiments of the present application, the anti-sticking agent is selected from the group consisting of talc powder, magnesium oxide, silicon dioxide, magnesium stearate, and combinations thereof. Alternatively, the anti-sticking agents is selected from the group consisting of talc powder and magnesium oxide.

In some embodiments of the present application, the pH-independent sustained-release layer comprises 50% to 80% by weigth of the sustained-release coating film-forming material, 5% to 30% by weigth of the plasticizer, 10% to 35% by weigth of the pore-forming agent, and 0% to 10% by weigth of the anti-sticking agent, based on a total amount of the sustained-release coating film-forming material, the plasticizer, the pore-forming agent, and the anti-sticking agent. A person skilled in the art can easily determine the content of the pH-independent sustained-release material in the sustained-release microspheres based on the desired sustained-release effect.

In some embodiments of the present application, the pH-dependent coating layer can be a pH-dependent coating layer with a pH ranging from about 5.5 to about 7.2. Those skilled in the art can understand that if a pH-dependent coating layer with a pH of about 5.5 is selected, then the drug delivery composition can typically target a duodenal site; if a pH-dependent coating layer with a pH of about 6.8 is selected, then the drug delivery composition can typically target a small intestinal site; and if a pH-dependent coating layer with a pH of about 7.0 is selected, then the drug delivery composition can typically target a colonic site. In some embodiments of the present application, the pH-dependent coating layer is a pH-dependent coating layer with a pH of about 7.0.

In some embodiments of the present application, the pH-dependent coating layer comprises a pH-dependent coating film-forming material and one or more pharmaceutically acceptable excipients selected from the group consisting of a plasticizer and an anti-sticking agent.

In some embodiments of the present application, the pH-dependent coating film-forming material is selected from the group consisting of polymethacrylate, hydroxypropyl methylcellulose phthalate, polyacrylic resin, hydroxypropyl methylcellulose acetate succinate, and combinations thereof. Alternatively, the pH-dependent coating film-forming material is selected from the group consisting of hydroxypropyl methylcellulose phthalate, polyacrylic resin, and combinations thereof. Alternatively, the pH-dependent coating film-forming material is polyacrylic resin.

In some embodiments of the present application, the polyacrylic resin is EUDRAGIT, which is a collective name for methacrylic acid copolymer and methacrylate copolymer. Examples of EUDRAGIT include EUDRAGIT RL-100, EUDRAGIT RS-100, EUDRAGIT E30D55, EUDRAGIT L30D-55, EUDRAGIT L-100, EUDRAGIT S-100, EUDRAGIT E 30D, and EUDRAGIT E-100. EUDRAGIT L-100 is dissolved in a solution with pH>6, while EUDRAGIT S-100 is dissolved in a solution with pH>7.

In some embodiments of the present application, a layer of a non-ionic coating material can be applied as a barrier coating layer prior to applying the pH-dependent coating layer to prevent penetration of moisture. If the pH-dependent coating layer is uniform and complete, then it can prevent penetration of moisture without the need for the barrier coating layer. The barrier coating layer comprises hydroxypropyl methylcellulose E5, hydroxypropyl methylcellulose E50, povidone K30, copovidone, Opadry, or combinations thereof.

In some embodiments of the present application, the plasticizer contained in the pH-dependent coating layer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof. Alternatively, the plasticizer is selected from the group consisting of triethyl citrate and glycerol triacetate. Alternatively, the plasticizer is triethyl citrate.

In some embodiments of the present application, the pH-dependent coating layer comprises polymethacrylate (such as EUDRAGIT L-100 and EUDRAGIT S-100) as the pH-dependent coating film-forming material, and one or more excipients selected from the group consisting of triethyl citrate, polyethylene glycol (such as PEG 6000), and diethyl phthalate, and dibutyl phthalate as the plasticizer.

In some embodiments of the present application, the anti-sticking agent contained in the pH-dependent coating layer comprises one or more selected from the group consisting of talc powder, magnesium oxide, silicon dioxide, and magnesium stearate.

In some embodiments of the present application, the pH-dependent coating layer comprises 50% to 90% by weigth of the pH-dependent coating film-forming material, 10% to 40% by weigth of the plasticizer, and 0 to 10% by weigth of the anti-sticking agent.

In some embodiments of the present application, the pH-dependent coating layer can be applied on the outer surface of the pH-independent sustained-release layer, or the pH-dependent coating layer can be applied on an outer surface of a tablet comprising a multi-particulate system containing the pH-independent sustained-release layer.

In some embodiments of the present application, the time-dependent pulsatile controlled-release layer can release the active ingredient after an appropriate time-lag, for example, a time-lag of 2 to 6 hours, 2 to 4 hours, or 3 to 4 hours.

In some embodiments of the present application, the time-dependent pulsatile controlled-release layer comprises a permeation control material and a permeation regulating material

In some embodiments of the present application, the permeation control material is one or both of glyceryl behenate and ethyl cellulose. In some embodiments of the present application, the permeation control material is glyceryl behenate, or a mixture of glyceryl behenate and ethyl cellulose.

Glyceryl behenate can be present in the form of monoester, diester, triester, or a mixture thereof. The HLB value of glyceryl behenate is preferably less than 5, and is more preferably about 2. The content of glyceryl behenate in the time-dependent pulsatile controlled-release layer is about 5% to 85% by weight, 10% to 70% by weight, or 30% to 50% by weight. Ethyl cellulose is a non-swellable polymeric material that is insoluble in water, an acid or a base. The content of ethyl cellulose in the time-dependent pulsatile controlled-release layer is 0.5% to 10% by weight or 3% to 5% by weight, provided that a total content of both glyceryl behenate and ethyl cellulose in the time-dependent pulsatile controlled-release layer is 10% to 90% by weight.

In some embodiments of the present application, the permeation regulating material is selected from the group consisting of polyethylene glycol, hydroxypropyl methylcellulose, copovidone, povidone, polyoxyethylene ether-glycerol butanediol ester, methyl silicone oil, zinc stearate, calcium stearate, aluminum stearate, sodium stearate, polysorbate, talc powder, silicon dioxide, and combinations thereof. Polysorbate, such as polysorbate 80, can adjust the hydrophilicity of the controlled-release layer. Talc powder belongs to a hydrophilic excipient, which not only regulates the hydrophilicity but also has an anti-sticking effect.

In some embodiments of the present application, the time-dependent controlled-release layer comprises 10% to 90% by weight of the permeation control material and 10% to 90% by weight of the permeation regulating material.

In some embodiments of the present application, the active ingredient comprised in the intestine-targeted drug delivery composition can be a hydrophilic or hydrophobic drug that requires administration to the intestinal tract, for example, a JAK inhibitor that requires administration to the intestinal tract, such as Ruxolitinib, Tofacitinib, Baricitinib, Upadacitinib,

Abrocitinib, Filgotinib, and salts thereof, and combinations thereof; and a hormone drug that requires administration to the intestinal tract, such as cortisone, hydrocortisone, prednisone, prednisolone, methylprednisolone, triamcinolone, dexamethasone, betamethasone, triamcinolone acetonide, and salts thereof, and combinations thereof.

In another aspect, the present application provides a method for preparing the intestine-targeted drug delivery composition, comprising the steps of:

### (1) Preparation of a pH-Independent Sustained-Release Layer

Adding an active ingredient, a sustained-release coating film-forming material, and one or more pharmaceutically acceptable excipients selected from the group consisting of a plasticizer, a pore-forming agent, and an anti-sticking agent to an aqueous solution of an alcohol to form a sustained-release coating solution, and then coating blank microsphere cores with the sustained-release coating solution, for example, with a coating weight increase by 20 wt % to 100 wt %, to obtain sustained-release microspheres; or

Adding a sustained-release coating film-forming material and one or more pharmaceutically acceptable excipients selected from the group consisting of a plasticizer, a pore-forming agent, and an anti-sticking agent to an aqueous solution of an alcohol to form a sustained-release coating solution; granulating an active ingredient, a binder, and a filler to obtain drug-containing microsphere cores, for example those with a particle size of 0.1 mm to 2 mm, 0.2 mm to 1 mm, or 0.2 mm to 0.6 mm; and then coating the drug-containing microsphere cores with the sustained-release coating solution, for example, with a coating weight increase by 20 wt % to 100 wt %, to obtain sustained-release microspheres; or

Adding a sustained-release coating film-forming material and one or more pharmaceutically acceptable excipients selected from the group consisting of a plasticizer, a pore-forming agent, and an anti-sticking agent to an aqueous solution of an alcohol to form a sustained-release coating solution; coating blank microsphere cores with a solution containing an active ingredient and a binder to obtain drug-containing microsphere cores; and then coating the drug-containing microsphere cores with the sustained-release coating solution, for example, with a coating weight increase by 20 wt % to 100 wt %, to obtain sustained-release microspheres; or

Granulating an active ingredient, a binder, a filler, and a pH-independent sustained-release material to obtain sustained-release microspheres containing the active ingredient and the pH-independent sustained-release material;

### (2) Preparation of a pH-Dependent Coating Layer

Adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the sustained-release microspheres obtained in step (1) with the pH-dependent coating solution, for example, with a coating weight increase by 5 wt % to 50 wt %, to obtain pH-dependent microspheres; or

Adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the sustained-release microspheres obtained in step (1) with the pH-dependent coating solution, for example, with a coating weight increase by 5 wt % to 50 wt %, to obtain pH-dependent microspheres; and mixing the pH-dependent microspheres with a filler, a disintegrant, and/or a lubricant, and then tableting to obtain a pH-dependent tablet, for example a pH-dependent tablet with a hardness of 30 to 100 N; or

Mixing the sustained-release microspheres obtained in step (1) with a filler, a disintegrant, and/or a lubricant, and then tableting to obtain a compressed tablet, for example a compressed tablet with a hardness of 30 to 100 N; and adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the compressed tablet with the pH-dependent coating solution, for example, with a coating weight increase by 5 wt % to 50 wt %, to obtain a pH-dependent tablet; and

### (3) Preparation of a Time-Dependent Pulsatile Controlled-Release Layer

Adding a permeation control material and a permeation regulating material to an aqueous solution of an alcohol to form a time-dependent coating solution, and then coating the pH-dependent microspheres or the pH-dependent tablet with the time-dependent coating solution, for example, with a coating weight increase by 50 wt % to 400 wt %, to obtain the intestine-targeted drug delivery composition in microsphere form or tablet form.

In some embodiments of the present application, the sustained-release coating film-forming material, the plasticizer, the pore-forming agent, and optional the anti-sticking agent are added to the aqueous solution of the alcohol in step (1).

In some embodiments of the present application, the pH-dependent coating film-forming material, the plasticizer, and optional the anti-sticking agent are added to the aqueous solution of the alcohol in step (2).

In some embodiments of the present application, the blank microsphere cores are microcrystalline cellulose microsphere cores, silicon dioxide microsphere cores, or sucrose microsphere cores. Alternatively, the blank microsphere cores are microcrystalline cellulose microsphere cores or silicon dioxide microsphere cores.

In some embodiments of the present application, the blank microsphere cores have a particle size of 0.1 mm to 2 mm, 0.2 mm to 1 mm, or 0.2 mm to 0.6 mm.

In some embodiments of the present application, the binder used in step (1) of the above preparation method is a commonly-used pharmaceutical binder in the art. Examples of the binder include, but are not limited to, starch slurry, hydroxypropyl cellulose, methyl cellulose, hydroxypropyl methylcellulose, sodium carboxymethyl cellulose, and combinations thereof.

In some embodiments of the present application, the filler is a commonly-used pharmaceutical filler in the art. Examples of the filler include, but are not limited to, microcrystalline cellulose, mannitol, sucrose, maltose, xylitol, lactose, glucose, starch, sorbitol, and combinations thereof. In some embodiments of the present application, the filler is microcrystalline cellulose and/or lactose.

In some embodiments of the present application, the disintegrant is a commonly-used pharmaceutical disintegrant in the art. Examples of the disintegrant include, but are not limited to, low substituted hydroxypropyl cellulose, croscarmellose sodium, crospovidone, carboxymethyl starch sodium, dry starch, and combinations thereof. In some embodiments of the present application, the disintegrant is selected from low substituted hydroxypropyl cellulose and/or croscarmellose sodium.

In some embodiments of the present application, the lubricant is a commonly-used pharmaceutical lubricant in the art. Examples of the lubricant include, but are not limited to, magnesium stearate, micronized silica gel, talc powder, hydrogenated vegetable oil, and combinations thereof. Preferably, the lubricant is magnesium stearate.

In some embodiments of the present application, the alcohol used in the preparation method is a commonly-used alcohol solvent in the art. Examples of the alcohol include, but are not limited to, ethanol and isopropanol. In some embodiments of the present application, the aqueous solution of the alcohol is usually a 95% ethanol aqueous solution, a 90% ethanol aqueous solution, a 95% isopropanol aqueous solution, a 90% isopropanol aqueous solution, a 5% ethanol aqueous solution, or a 5% isopropanol aqueous solution.

In still another aspect, the present application provides a use of the intestine-targeted drug delivery composition in the manufacture of a medicament for treating intestinal diseases, such as inflammatory bowel diseases including Crohn's disease and ulcerative colitis.

In a further aspect, the present application provides a method for treating intestinal diseases, such as inflammatory bowel diseases including Crohn's disease and ulcerative colitis, comprising administering to a subject in need thereof the intestine-targeted drug delivery composition.

The intestine-targeted drug delivery composition of the present application can disintegrate by absorbing water after about 2 to 6 hours, 2 to 4 hours, or 3 to 4 hours following oral administration, safely pass through gastric juice and reach the small intestine, and then release the active ingredient upon entry into the small intestine. Compared with existing oral formulations, the intestine-targeted drug delivery composition of the present application has one or more of the following advantages: allowing accurate site-specific delivery in intestinal tissues (such as colon tissue), reducing the systemic blood concentration of the active ingredient and reducing blood exposure thereof, improving the pharmacokinetics of the active ingredient, increasing the concentration of the active ingredient in intestinal tissues (such as colon tissue), enhancing the therapeutic efficacy, improving safety, and reducing systemic toxicity, side effects, and adverse reactions.

### EXAMPLES

The present application will be described in further details below in conjunction with specific examples. These examples may be modified to obtain other embodiments without departing from the scope or spirit of the present application. Therefore, the following examples are non-limitative.

Unless otherwise specified, all numbers used in the Specification and Claims to represent feature sizes, quantities, and physicochemical properties should be understood as being modified by the term “about” in all cases. Therefore, unless otherwise stated to the contrary, the numerical parameters listed in the foregoing Specification and the appended claims are approximations, and those skilled in the art can utilize the teachings disclosed herein to seek the desired characteristics, and appropriately change these approximations. The use of a numerical range represented by endpoints includes all numbers within said range and any range within said range, for example, 1 to 5 includes 1, 1.1, 1.3, 1.5, 2, 2.75, 3, 3.80, 4, and 5, etc.

The drugs or reagents used in the present application are all conventional commercially-available products, unless otherwise specified. For example, hydroxypropyl methylcellulose E5 was purchased from Dow Chemical Company, USA; microcrystalline cellulose PH-101, microcrystalline cellulose PH-102, low substituted hydroxypropyl cellulose SH-LH21, microcrystalline cellulose SH-101, microcrystalline cellulose SH-102, and low substituted hydroxypropyl cellulose SH-LH31 were purchased from Anhui Sunhere Pharmaceutical Excipients Co., Ltd.; Eudragit L100, Eudragit S100, and Eudragit L30D-55 were purchased from Roehm Chemical (Shanghai) Co., Ltd.; and Opadry film coating premix was purchased from Shanghai Colorcon Coating Technology Ltd.

### Comparative Example 1 (Tablets Containing Only pH-Independent Sustained-Release Layer)

5 g of tofacitinib citrate, 8 g of ethyl cellulose, 2 g of triethyl citrate, and 3g of hydroxypropyl methylcellulose E5 were weighed and added to 150 g of 90% ethanol at room temperature, stirred and dispersed uniformly to prepare a sustained-release coating solution. 34 g of microcrystalline cellulose blank microsphere cores were poured into a fluidized bed, and coated with the sustained-release coating solution to prepare drug-containing sustained-release microspheres.

The drug-containing sustained-release microspheres, 38 g of microcrystalline cellulose PH-101, 38 g of microcrystalline cellulose PH-102, 14 g of low substituted hydroxypropyl cellulose SH-LH21, and 1g of magnesium stearate were mixed uniformly. A φ6.5 mm circular deep concave punch was mounted on a tabletting machine, and tabletting was carried out to obtain tablets with a hardness of 40±10 N.

### Comparative Example 2 (Tablets Containing Only pH-Dependent Coating Layer)

5 g of tofacitinib citrate and an appropriate amount of hydroxypropyl methylcellulose E5 were weighed and added to 150 g of 90% ethanol at room temperature, stirred and dispersed uniformly to prepare a drug-containing solution. 34 g of microcrystalline cellulose blank microsphere cores were poured into a fluidized bed, and then coated by spraying the drug-containing solution to obtain drug-containing microsphere cores.

4 g of Eudragit L100, 4 g of Eudragit S100, and 3 g of triethyl citrate were weighed and added to 150 g of 95% isopropanol, stirred and dispersed uniformly to prepare a pH-dependent coating solution. The drug-containing microsphere cores were poured into a fluidized bed, and coated with the pH-dependent coating solution to obtain pH-dependent microspheres.

The pH-dependent microspheres, 38 g of microcrystalline cellulose PH-101, 38 g of microcrystalline cellulose PH-102, 14 g of low substituted hydroxypropyl cellulose SH-LH21, and 1 g of magnesium stearate were mixed uniformly. A Φ6.5 mm circular deep concave punch was mounted on a tabletting machine, and tabletting was carried out to obtain tablets with a hardness of 40±10 N.

### Comparative Example 3 (Tablets Containing Only Time-Dependent Pulsatile Controlled-Release Layer)

5 g of tofacitinib citrate and an appropriate amount of hydroxypropyl methylcellulose E5 were weighed and added to 150 g of 90% ethanol at room temperature, stirred and dispersed uniformly to prepare a drug-containing solution. 34 g of microcrystalline cellulose blank microsphere cores were poured into a fluidized bed, and coated by spraying the drug-containing solution to obtain drug-containing microsphere cores.

The drug-containing microsphere cores, 38 g of microcrystalline cellulose PH-101, 38 g of microcrystalline cellulose PH-102, 14 g of low substituted hydroxypropyl cellulose SH-LH21, and 1 g of magnesium stearate were mixed uniformly. A φ6.5 mm circular deep concave punch was mounted on a tabletting machine, and tabletting was carried out to obtain compressed tablets with a hardness of 40±10 N.

10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare tablets containing a time-dependent pulsatile controlled-release layer.

### Example 1

5 g of Tofacitinib citrate, 8 g of ethyl cellulose, 2 g of triethyl citrate, and 3 g of hydroxypropyl methylcellulose E5 were weighed and added to 150 g of 90% ethanol at room temperature, stirred and dispersed uniformly to prepare a sustained-release coating solution. 34 g of microcrystalline cellulose blank microsphere cores were poured into a fluidized bed, and coated with the sustained-release coating solution to prepare drug-containing sustained-release microspheres.

4 g of Eudragit L100, 4 g of Eudragit S100, and 3 g of triethyl citrate were weighed and added to 150 g of 95% isopropanol, stirred and dispersed uniformly to prepare a pH-dependent coating solution. The drug-containing sustained-release microspheres were poured into a fluidized bed, and coated with the pH-dependent coating solution to prepare pH-dependent microspheres.

The pH-dependent microspheres, 38 g of microcrystalline cellulose PH-101, 38 g of microcrystalline cellulose PH-102, 14 g of low substituted hydroxypropyl cellulose SH-LH21, and 1 g of magnesium stearate were mixed uniformly. A φ6.5 mm circular deep concave punch was mounted on a tabletting machine, and tabletting was carried out to obtain compressed tablets with a hardness of 40±10 N.

10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare an intestine-targeted pharmaceutical fomulation.

### Example 2

8 g of cellulose acetate, 1.5 g of triethyl citrate, and 3 g of hydroxypropyl methylcellulose E5 were weighed and added to 170 g of 90% ethanol at room temperature, stirred and dispersed uniformly to prepare a sustained-release coating solution. Drug-containing microsphere cores were prepared through wet-granulation and extrusion-spheronization of 5 g of tofacitinib citrate, 4 g of hydroxypropyl methylcellulose E5, and 30 g of microcrystalline cellulose. The drug-containing microsphere cores were poured into a fluidized bed, and coated with the sustained-release coating solution to obtain drug-containing sustained-release microspheres.

4 g of Eudragit L100, 4 g of Eudragit S100, and 3 g of triethyl citrate were weighed and added to 150 g of 95% isopropanol, stirred and dispersed uniformly to prepare a pH-dependent coating solution. The drug-containing sustained-release microspheres were poured into a fluidized bed, and coated with the pH-dependent coating solution to prepare pH-dependent microspheres.

The pH-dependent microspheres, 30 g of microcrystalline cellulose PH-101, 30 g of microcrystalline cellulose PH-102, 10 g of low substituted hydroxypropyl cellulose SH-LH21, and 1 g of magnesium stearate were mixed uniformly. A φ6.5 mm circular deep concave punch was mounted on a tabletting machine, and tabletting was carried out to obtain compressed tablets with a hardness of 40±10 N.

12 g of ethyl cellulose, 25 g of hydroxypropyl methylcellulose E15, 9 g of polysorbate 80, 150 g of glyceryl behenate, and 100 g of talc powder were weighed and added to 2 L of 90% isopropanol, stirred and dispersed uniformly to prepare a coating solution. The compressed tablets were poured into a high-performance coating pot, and coated with the coating solution to prepare a colon-targeted pharmaceutical formulation.

### Example 3

8 g of cellulose acetate, 1.5 g of triethyl citrate, and 3 g of hydroxypropyl methylcellulose E5 were weighed and added to 170 g of 90% ethanol at room temperature, stirred and dispersed uniformly to prepare a sustained-release coating solution. An aqueous suspension containing 5 g of tofacitinib citrate and 4 g of copovidone was sprayed on microcrystalline cellulose microsphere cores to prepare drug-containing microsphere cores. The drug-containing microsphere cores were poured into a fluidized bed, and coated with the sustained-release coating solution to obtain drug-containing sustained-release microspheres.

4 g of Eudragit L100, 4 g of Eudragit E100, and 3 g of triethyl citrate were weighed and added to 150 g of 95% isopropanol, stirred and dispersed uniformly to prepare a pH-dependent coating solution. The drug-containing sustained-release microspheres were poured into a fluidized bed, and coated with the pH-dependent coating solution to prepare pH-dependent microspheres.

The pH-dependent microspheres, 35 g of microcrystalline cellulose SH-101, 35 g of microcrystalline cellulose SH-102, 15 g of low substituted hydroxypropyl cellulose SH-LH31, and 1 g of magnesium stearate were mixed uniformly. A φ6.5 mm circular deep concave punch was mounted on a tabletting machine, and tabletting was carried out to obtain compressed tablets with a hardness of 40±10 N.

10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare a colon-targeted pharmaceutical formulation.

### Example 4

5 g of Tofacitinib citrate, 8 g of ethyl cellulose, 2 g of triethyl citrate, and 3 g of hydroxypropyl methylcellulose E5 were weighed and added to 150 g of 90% ethanol at room temperature, stirred and dispersed uniformly to prepare a sustained-release coating solution. 34 g of microcrystalline cellulose microsphere cores were poured into a fluidized bed, and coated with the sustained-release coating solution to prepare drug-containing sustained-release microspheres.

The drug-containing sustained-release microspheres, 38 g of microcrystalline cellulose PH-101, 38 g of microcrystalline cellulose PH-102, 14 g of low substituted hydroxypropyl cellulose SH-LH31, and 1 g of magnesium stearate were mixed uniformly. A φ6.5 mm circular deep concave punch was mounted on a tabletting machine, and tabletting was carried out to obtain compressed tablets with a hardness of 40±10 N.

4 g of Eudragit L100, 4 g of Eudragit S100, and 3 g of triethyl citrate were weighed and added to 150 g of 95% isopropanol, stirred and dispersed uniformly to prepare a pH-dependent coating solution. The compressed tablets were poured into a fluidized bed, and coated with the pH-dependent coating solution to prepare pH-dependent tablets.

10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The pH-dependent tablets were poured into a high-performance coating pot, and coated with the coating solution to prepare a colon-targeted pharmaceutical formulation.

### Example 5

5 g of Tofacitinib citrate, 8 g of ethyl cellulose, 3 g of hydroxypropyl methylcellulose E5, and 30 g of microcrystalline cellulose PH-101 were weighed and poured into a wet mixing granulator at room temperature, to which 20% ethanol was added by spraying to prepare a wet soft material. The wet soft material was subjected to extrusion-spheronization to prepare drug-containing sustained-release microspheres.

4 g of Eudragit L100, 4 g of Eudragit S100, and 3 g of triethyl citrate were weighed and added to 150 g of 95% isopropanol, stirred and dispersed uniformly to prepare a pH-dependent coating solution. The drug-containing sustained-release microspheres were poured into a fluidized bed, and coated with the pH-dependent coating solution to prepare pH-dependent microspheres.

The pH-dependent microspheres, 38 g of microcrystalline cellulose PH-101, 38 g of microcrystalline cellulose PH-102, 14 g of low substituted hydroxypropyl cellulose SH-LH21, and 1g of magnesium stearate were mixed uniformly. A φ6.5 mm circular deep concave punch was mounted on a tabletting machine, and tabletting was carried out to obtain compressed tablets with a hardness of 40±10 N.

10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare an intestine-targeted pharmaceutical formulation.

### Example 6

Compressed tablets were prepared by the same operations as in Example 1.

1 g of Allura red, 8 g of ethyl cellulose, 18 g of hydroxypropyl methylcellulose E5, 9 g of polysorbate 80, 130 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare a colon-targeted pharmaceutical formulation.

### Example 7

Compressed tablets were prepared by the same operations as in Example 1.

1 g of Allura red, 10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare a colon-targeted pharmaceutical formulation.

### Example 8

Compressed tablets were prepared by the same operations as in Example 1.

1 g of Allura red, 12 g of ethyl cellulose, 18 g of hydroxypropyl methylcellulose E5, 9 g of polysorbate 80, 230 g of glyceryl behenate, and 129 g of talc powder were weighed and added to 2 kg of 90% isopropanol, stirred and dispersed uniformly to prepare a coating solution.

The compressed tablets were poured into a high-performance coating pot, and coated with the coating solution to prepare a colon-targeted pharmaceutical formulation.

### Example 9

5 g of Upadacitinib, 8 g of ethyl cellulose, 2 g of triethyl citrate, and 3 g of hydroxypropyl methylcellulose E5 were weighed and added to 150 g of 90% ethanol at room temperature, stirred and dispersed uniformly to prepare a sustained-release coating solution. 34 g of microcrystalline cellulose blank microsphere cores were poured into a fluidized bed, and coated with the sustained-release coating solution to prepare drug-containing sustained-release microspheres.

4 g of Eudragit L100, 4 g of Eudragit S100, and 3 g of triethyl citrate were weighed and added to 150 g of 95% isopropanol, stirred and dispersed uniformly to prepare a pH-dependent coating solution. The drug-containing sustained-release microspheres were poured into a fluidized bed, and coated with the pH-dependent coating solution to prepare pH-dependent microspheres.

The pH-dependent microspheres, 38 g of microcrystalline cellulose PH-101, 38 g of microcrystalline cellulose PH-102, 14 g of low substituted hydroxypropyl cellulose SH-LH21, and 1 g of magnesium stearate were mixed uniformly. A φ6.5 mm circular deep concave punch was mounted on a tabletting machine, and tabletting was carried out to obtain compressed tablets with a hardness of 40 +10 N.

10 g of ethyl cellulose, 18 g of hydroxypropyl methylcellulose E5, 9 g of polysorbate

80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare a colon-targeted pharmaceutical formulation.

### Example 10

8 g of prednisolone, 8 g of ethyl cellulose, 2 g of triethyl citrate, and 3 g of hydroxypropyl methylcellulose E5 were weighed and added to 150 g of 90% ethanol at room temperature, stirred and dispersed uniformly to prepare a sustained-release coating solution. 34g of microcrystalline cellulose blank microsphere cores were poured into a fluidized bed, and coated with the sustained-release coating solution to prepare drug-containing sustained-release microspheres.

Application of barrier coating: The Opadry thin film coating premix and purified water were weighed. The coating powder was added to the purified water under stirring to prepare a coating solution with a concentration of 15% (W/W). The drug-containing sustained-release microspheres were coated with the coating solution to obtain a coating weight increase by about 3%. During the coating process, the temperature of the tablet bed was controlled at 40±2° C. After the spraying was finished, the inlet air temperature was set to 60±10° C. and the rotation speed of the coating pot was set to 1 to 3 rpm.

30 g of Eudragit L30D-55, 5 g of triethyl citrate, and 15 g of talc powder were weighed and added to 150 g of 5% isopropanol, stirred and dispersed uniformly to prepare a pH-dependent coating solution. The drug-containing sustained-release microspheres that had been coated with the barrier coating were poured into a fluidized bed, and coated with the pH-dependent coating solution to prepare pH-dependent microspheres.

The pH-dependent microspheres, 38 g of microcrystalline cellulose PH-101, 38 g of microcrystalline cellulose PH-102, 14 g of low substituted hydroxypropyl cellulose SH-LH21, and 1g of magnesium stearate were mixed uniformly. A φ6.5 mm circular deep concave punch was mounted on a tabletting machine, and tabletting was carried out to obtain compressed tablets with a hardness of 40±10N.

10 g of ethyl cellulose, 18 g of hydroxypropyl methylcellulose E5, 9 g of polysorbate 80, 178 g of glyceryl behenate, and 129 g of talc powder were weighed and added to 2 kg of 90% isopropanol, stirred and dispersed uniformly to prepare a coating solution. The compressed tablets were poured into a high-performance coating pot, and coated with the coating solution to prepare intestine-targeted pharmaceutical formulation.

### Performance Tests

As used herein, a method for determining the dissolution and release in vitro is described as follows: a product was tested according to the dissolution and release test method specified in Method 1 in Appendix 0931 of Part IV of the Chinese Pharmacopoeia, Edition 2020, in which 900 ml of a phosphate buffer solution with pH 5.5 was used as a dissolution medium for the first 3 hours, and then 900 ml of a phosphate buffer solution with pH 7.2 was used as the dissolution medium for release until complete dissolution. The test was operated at a speed of 100 rpm according to the above method. During the test, 5 ml of each solution was taken and filtered after 3 hours, 4 hours, 5 hours, 7 hours, 9 hours, 11 hours, 15 hours, and 19 hours, and the subsequent filtrate was taken. The release degree in the different dissolution media was determined according to the high-performance liquid chromatography method specified in Appendix 0512 of Part IV of the Chinese Pharmacopoeia, Edition 2020. A 3-hour release amount in the phosphate buffer solution with pH 5.5 should not exceed 2%; a 4-hour release amount in the phosphate buffer solution with pH 7.2 should be 15% to 30%; a 7-hour release amount should be 40% to 60%; and a 19-hour release amount should be 75% or more.

Unless otherwise specified, the amount of tofacitinib was detected using the following high-performance liquid chromatography conditions:

Octadecylsilane-bonded silica gel was used as a filler; a phosphate buffer solution with pH 6.8 (about 1.3 g of KH₂PO₄ and 5 ml of triethylamine were added to 1000 ml of water, and if necessary, adjusted to pH 6.8 with H₃PO₄)—acetonitrile (70:30) was used as the mobile phase; the flow rate was 1.0 ml per minute; the detection wavelength was 289 nm; the column temperature was 35° C.; and the injection volume was 20 μl.

In vitro dissolution and in vivo pharmacokinetic tests were conducted on the pharmaceutical formulations prepared in the Comparative Examples and Examples using the methods described above and below. The experimental results were shown below.

### Test on Comparative Examples 1-3 and Example 1

### (1) Dissolution Curve Determination Results

TABLE 1
In vitro dissolution determination results
of Comparative Examples 1-3 and Example 1

| | Time (h) | | | | | | | | |
|---|---|---|---|---|---|---|---|---|---|
| No. | 0 | 3 | 4 | 5 | 7 | 9 | 11 | 15 | 19 |
| Comparative | 0 | 35.25 | 44.53 | 52.36 | 66.55 | 75.98 | 81.34 | 86.35 | 88.56 |
| Example 1 | | | | | | | | | |
| Comparative | 0 | 5.66 | 92.56 | 93.45 | 93.4 | 93.69 | 94.05 | 93.95 | — |
| Example 2 | | | | | | | | | |
| Comparative | 0 | 0.11 | 90.21 | 91.56 | 92.51 | 92.15 | 93.05 | 93.46 | — |
| Example 3 | | | | | | | | | |
| Example 1 | 0 | 0.37 | 22.18 | 35.64 | 51.69 | 63.98 | 73.60 | 85.35 | 92.24 |

### (2) Pharmacokinetics

This study adopted a regimen of a single administration of a single dose. 48 healthy Beagle dogs (24 males and 24 females with a body weight in the range of 12 kg to 16 kg at the time of administration) were selected and randomly divided into 4 groups, and each group received an oral administration. Blood was collected and animals in each group were dissected at each time point after administration. The administration regimen was shown in Table 2.

TABLE 2
Administration regimen

| | | | Quantity of |
|---|---|---|---|
| Group | | | animals |
| No. | Test sample | Dosage | Female/male |
| 1 | Comparative Example 1 | 5 mg/animal ^{a} | 6/6 |
| 2 | Comparative Example 2 | 5 mg/animal ^{a} | 6/6 |
| 3 | Comparative Example 3 | 5 mg/animal ^{a} | 6/6 |
| 4 | Example 1 | 5 mg/animal ^{a} | 6/6 |

^{a} indicates that the administration concentration was calculated based on the free base of tofacitinib.

### Collection of Intestinal Tissues and Contents

Sampling frequency and time points: at each time point as shown in the table below, blood was collected from the veins of the limbs, the animals were anesthetized with Zoletil 50 and xylazine hydrochloride immediately after the blood collection, and subsequently sacrificed by the femoral artery exsanguinating, and intestinal tissues, such as the duodenum, jejunum, ileum, cecum, and colorectum, and contents thereof were collected.

Sample count: All animals.

| | | Animals at | Time point of blood |
|---|---|---|---|
| Group | | each time | collection and dissection |
| No. | Test sample | point | (h) |
| 1 | Comparative Example 1 | 3 | 1 h, 2 h, 4 h, 12 h |
| 2 | Comparative Example 2 | 3 | 1 h, 2 h, 4 h, 12 h |
| 3 | Comparative Example 3 | 3 | 1 h, 2 h, 4 h, 12 h |
| 3 | Example 1 | 3 | 1 h, 2 h (blood collection |
| | | | only), 4 h, 8 h, 12 h |

### Separation and Preservation of Samples

Sampling requirements and treatment of blood samples: about 2 mL of blood was collected from the veins of the limbs using an EDTA-K₂ anticoagulant tube each time, and the plasma of the collected blood was separated through centrifugation as soon as possible after the blood was collected. The plasma was firstly frozen and temporarily stored at about −20° C., and then transferred to a sample room and stored in a refrigerator for further testing and analysis.

After the tissues and contents were collected, the contents were washed with physiological saline, and then frozen at −20° C. Before analysis, tissue homogenate was prepared using the homogenization method for analysis.

### Biological Sample Analysis

After sampling, the drug concentration of tofacitinib in the sample was detected by

the LC-MS/MS method.

### Detection Conditions

Detection system: LC-MS/MS liquid chromatography tandem mass spectrometry (Thermo TSQ Quantem Discovery MAX)
Liquid chromatography column: Luna 3 μm C18, S/N: H19-302624
Column temperature: 40° C.
Autosampler temperature: room temperature
Mobile phase A: 0.1% aqueous solution of formic acid (pH 6)
Mobile phase B: methanol
Needle wash: methanol: isopropanol: water (1:1:1)

Liquid phase ratio:

| Time (min) | A % | B % | μL/min |
|---|---|---|---|
| 0 | 65 | 35 | 500 |
| 2 | 0 | 100 | 500 |
| 2.5 | 0 | 100 | 500 |
| 2.51 | 65 | 35 | 500 |
| 3.5 | 65 | 35 | 500 |

Ionization method: ESI+
Scan mode: SRM

For quantitative analysis of precursor ions,
fragment ions, and collision energy (CE)

| | Precursor | Product | | Q1 | Q3 | Tube |
|---|---|---|---|---|---|---|
| Compound | (m/z) | (m/z) | CE(eV) | PW | PW | L |
| Tofacitinib citrate | 313.2 | 149.1 | 29 | 0.7 | 0.7 | 113 |
| Labetalol (internal | 329.1 | 162.0 | 26 | 0.7 | 0.7 | 113 |
| standard) | | | | | | |

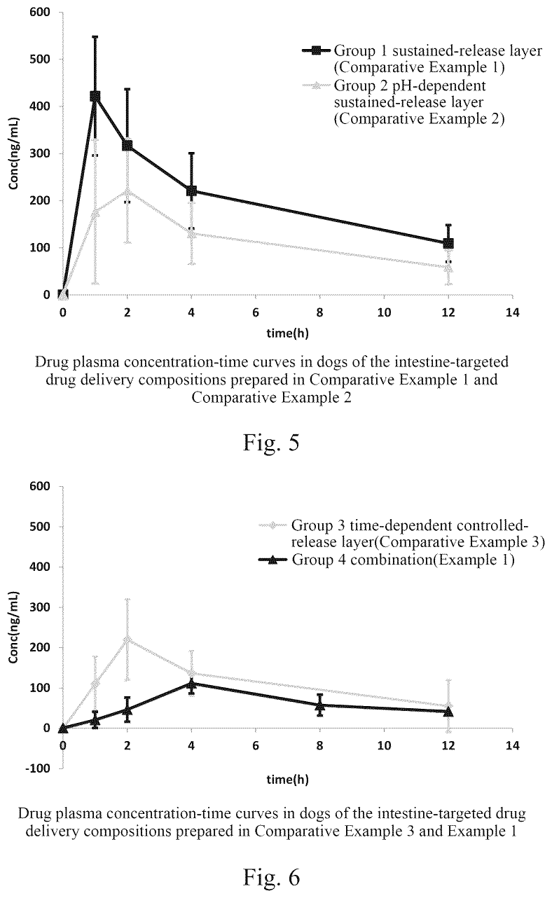

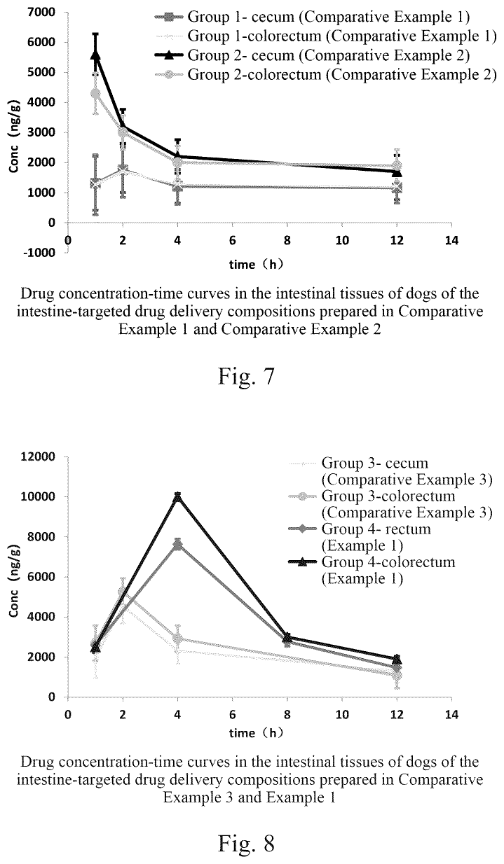

The experimental results shown in FIG. 5 to FIG. 8 indicated that, compared to the intestine-targeted pharmaceutical formulations in Comparative Examples 1 to 3, the intestine-targeted pharmaceutical formulation in Example 1 can be accurately delivered to intestinal tissues, especially the colorectal tissue, to release the active ingredient.

### Test on Examples 1 to 4

### (1) Dissolution Curve Determination Results

TABLE 3
Dissolution determination results of Examples 1-4

| | Time(h) | | | | | | | | |
|---|---|---|---|---|---|---|---|---|---|
| No. | 0 | 3 | 4 | 5 | 7 | 9 | 11 | 15 | 19 |
| Example 1 | 0 | 0.37 | 22.18 | 35.64 | 51.69 | 63.98 | 73.60 | 85.35 | 92.24 |
| Example 2 | 0 | 0 | 22.85 | 34.25 | 47.99 | 58.59 | 67.00 | 78.52 | 85.46 |
| Example 3 | 0 | 0 | 19.23 | 32.40 | 46.53 | 59.23 | 65.10 | 77.34 | 84.78 |
| Example 4 | 0 | 0.22 | 15.96 | 30.41 | 50.15 | 63.85 | 73.78 | 84.65 | 90.42 |

### (2) Pharmacokinetics

### Experimental Design

This study adopted a regimen of a single administration of a single dose. 30 healthy Beagle dogs (15 males and 15 females with a body weight in the range of 12 kg to 16 kg at the time of administration) were selected and randomly divided into 4 groups, and the dogs in each group were orally administered with the intestine-targeted pharmaceutical formulations in Examples 1, 2, and 4 and the conventional tablet of tofacitinib citrate (control drug, tofacitinib citrate tablet, batch No.: DE8535, specification: 5 mg, Pfizer). Blood was collected and animals in each group were dissected at each time point after oral administration. The administration regimen was shown in Table 4.

TABLE 4
Administration regimen

| | | | Quantity of animals |
|---|---|---|---|
| Group | Test sample | Dosage | Female/male |
| 1 | Example 1 | 5 mg/animal ^{a} | 5/5 |
| 2 | Example 2 | 5 mg/animal ^{a} | 2/2 |
| 3 | Example 4 | 5 mg/animal ^{a} | 2/2 |
| 4 | Tofacitinib citrate tablet | 5 mg/animal ^{a} | 6/6 |

^{a} indicates that the administration concentration was calculated based on the free base of tofacitinib.

### Collection of Intestinal Tissues and Contents

Sampling frequency and time points: at each time point as shown in the table below, blood was collected from the veins of the limbs, the animals were anesthetized with Zoletil 50 and xylazine hydrochloride immediately after the blood collection, and subsequently sacrificed by the femoral artery exsanguinating, and intestinal tissues, such as the duodenum, jejunum, ileum, cecum, and colorectum, and contents thereof were collected.

Sample count: All animals.

| Group | | Animals at | Time point of blood |
|---|---|---|---|
| No. | Test sample | each time point | collection and dissection (h) |
| 1 | Example 1 | 2 | 1 h, 2 h, 4 h, 8 h, 12 h |
| 2 | Example 2 (blood | 2 | 1 h, 2 h, 4 h, 8 h, 12 h |
| | collection only) | | |
| 3 | Example 4 (blood | 2 | 1 h, 2 h, 4 h, 8 h, 12 h |
| | collection only) | | |
| 4 | Tofacitinib citrate | 2 | 0.5 h, 1 h, 2 h, 4 h, 8 h, |
| | tablet | | 12 h |

### Separation and Preservation of Samples

Sampling requirements and treatment of blood samples: about 2 mL of blood was collected from the veins of the limbs using EDTA-K₂ anticoagulant tubes each time, and the plasma of the collected blood was separated through centrifugation as soon as possible after the blood was collected. The plasma was firstly frozen and temporarily stored at about −20° C., and then transferred to a sample room and stored in a refrigerator for further testing and analysis.

After the tissues and contents were collected, the contents were washed with physiological saline, and then frozen at −20° C. Before analysis, tissue homogenate was prepared using the homogenization method for analysis.

### Biological Sample Analysis

After sampling, the drug concentration of tofacitinib in the sample was detected by the LC-MS/MS method.

### Detection Conditions

Detection system: LC-MS/MS liquid chromatography tandem mass spectrometry (Thermo TSQ Quantem Discovery MAX)
Liquid chromatography column: Luna 3 μm C18, S/N: H19-302624
Column temperature: 40° C.
Autosampler temperature: room temperature
Mobile phase A: 0.1% aqueous solution of formic acid (pH 6)
Mobile phase B: methanol
Needle wash: methanol: isopropanol: water (1:1:1)

Liquid phase ratio:

| Time (min) | A % | B % | μL/min |
|---|---|---|---|
| 0 | 65 | 35 | 500 |
| 2 | 0 | 100 | 500 |
| 2.5 | 0 | 100 | 500 |
| 2.51 | 65 | 35 | 500 |
| 3.5 | 65 | 35 | 500 |

Ionization method: ESI+
Scan mode: SRM

For quantitative analysis of precursor ions,
fragment ions, and collision energy (CE)

| | Precursor | Product | | Q1 | Q3 | Tube |
|---|---|---|---|---|---|---|
| Compound | (m/z) | (m/z) | CE(eV) | PW | PW | L |
| Tofacitinib citrate | 313.2 | 149.1 | 29 | 0.7 | 0.7 | 113 |
| Labetalol (internal | 329.1 | 162.0 | 26 | 0.7 | 0.7 | 113 |
| standard) | | | | | | |

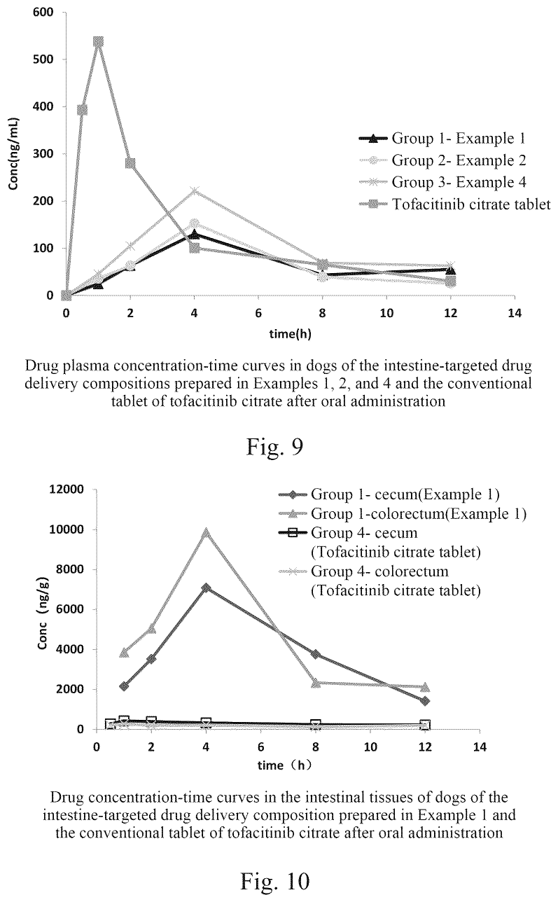

The experimental results shown in FIG. 9 to FIG. 10 indicated that, the local concentration of the intestine-targeted pharmaceutical formulation in Example 1 in the intestinal tissues, especially in the colorectal tissues, was higher than that of the conventional tablet of tofacitinib citrate after oral administration; and the plasma exposure amounts of the intestine-targeted pharmaceutical formulations in Examples 1, 2, and 4 were lower than that of the conventional tablet of tofacitinib citrate after oral administration.

### Test on Examples 6-8

### (1) Observation of Dissolution Phenomenon

900 ml of a phosphate buffer solution with pH 7.2 was used as the medium, the basket method was performed for 100 revolutions, and the disintegration time of the time-delay coating was observed. (Allura red was contained in the time-delay coating)

| No. | Time (h) |
|---|---|
| Example 6 | 1.75 h |
| Example 7 | 3.17 h |
| Example 8 | 4.5 h |

What is claimed is:

1. An intestine-targeted drug delivery composition, comprising in sequence, from inside to outside, (1) a microsphere core and a pH-independent sustained-release layer containing an active ingredient, or a microsphere containing an active ingredient and a pH-independent sustained-release layer, or a microsphere containing an active ingredient and a pH-independent sustained-release material, (2) a pH-dependent coating layer, and (3) a time-dependent pulsatile controlled-release layer.

2. The composition according to claim 1, wherein the pH-independent sustained-release layer comprises a sustained-release coating film-forming material and one or more pharmaceutically acceptable excipients selected from the group consisting of a plasticizer, a pore-forming agent, and an anti-sticking agent; or the pH-independent sustained-release layer comprises a sustained-release coating film-forming material, a plasticizer, and one or more pharmaceutically acceptable excipients selected from the group consisting of a pore-forming agent and an anti-sticking agent; or the pH-independent sustained-release layer comprises a sustained-release coating film-forming material, a pore-forming agent, and one or more pharmaceutically acceptable excipients selected from the group consisting of a plasticizer and an anti-sticking agent; or the pH-independent sustained-release layer comprises a sustained-release coating film-forming material, a plasticizer, and a pore-forming agent, and optionally an anti-sticking agent.

3. The composition according to claim 1, wherein the pH-dependent coating layer is a pH-dependent coating layer with pH ranging from 5.5 to 7.2, and comprises a pH-dependent coating film-forming material and one or more pharmaceutically acceptable excipients selected from the group consisting of a plasticizer and an anti-sticking agent, or comprises a pH-dependent coating film-forming material and a plasticizer, and optionally an anti-sticking agent.

4. The composition according to claim 2, wherein the pH-dependent coating layer is a pH-dependent coating layer with pH ranging from 5.5 to 7.2, and comprises a pH-dependent coating film-forming material and one or more pharmaceutically acceptable excipients selected from the group consisting of a plasticizer and an anti-sticking agent, or comprises a pH-dependent coating film-forming material and a plasticizer, and optionally an anti-sticking agent.

5. The composition according to claim 1, wherein the time-dependent pulsatile controlled-release layer can make the active ingredient release after a time-lag of 2 to 6 hours, 2 to 4 hours, or 3 to 4 hours, and the time-dependent pulsatile controlled-release layer comprises a permeation control material and a permeation regulating material.

6. The composition according to claim 2, wherein the time-dependent pulsatile controlled-release layer can make the active ingredient release after a time-lag of 2 to 6 hours, 2 to 4 hours, or 3 to 4 hours, and the time-dependent pulsatile controlled-release layer comprises a permeation control material and a permeation regulating material.

7. The composition according to claim 3, wherein the time-dependent pulsatile controlled-release layer can make the active ingredient release after a time-lag of 2 to 6 hours, 2 to 4 hours, or 3 to 4 hours, and the time-dependent pulsatile controlled-release layer comprises a permeation control material and a permeation regulating material.

8. The composition according to claim 4, wherein the time-dependent pulsatile controlled-release layer can make the active ingredient release after a time-lag of 2 to 6 hours, 2 to 4 hours, or 3 to 4 hours, and the time-dependent pulsatile controlled-release layer comprises a permeation control material and a permeation regulating material.

9. The composition according to claim 2,
wherein the sustained-release coating film-forming material is selected from the group consisting of methacrylate copolymer, ethyl cellulose, hydroxypropyl methylcellulose, cellulose acetate, and combinations thereof; or
wherein the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof; or
wherein the pore-forming agent is selected from the group consisting of polyethylene glycol, hydroxypropyl methylcellulose, copovidone, povidone, and combinations thereof; or
wherein the anti-sticking agent is selected from the group consisting of talc powder, magnesium oxide, silicon dioxide, magnesium stearate, and combinations thereof; or
wherein the pH-independent sustained-release material is selected from the group consisting of methacrylate copolymer, ethyl cellulose, hydroxypropyl methylcellulose, cellulose acetate, and combinations thereof.

10. The composition according to claim 3,
wherein the pH-dependent coating film-forming material is selected from the group consisting of polymethacrylate, hydroxypropyl methylcellulose phthalate, polyacrylic acid resin, hydroxypropyl methylcellulose acetate succinate, and combinations thereof; or
wherein the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof; or
wherein the anti-sticking agent is selected from the group consisting of talc powder, magnesium oxide, silicon dioxide, magnesium stearate, and combinations thereof.

11. The composition according to claim 5,
wherein the permeation control material is selected from glyceryl behenate and/or ethyl cellulose; or
wherein the permeation regulating material is selected from the group consisting of polyethylene glycol, hydroxypropyl methylcellulose, copovidone, povidone, polyoxyethylene ether glycerol butanediol ester, methyl silicone oil, zinc stearate, calcium stearate, aluminum stearate, sodium stearate, polysorbate, talc powder, silicon dioxide, and combinations thereof.

12. The composition according to claim 4,
wherein the sustained-release coating film-forming material is selected from the group consisting of methacrylate copolymer, ethyl cellulose, hydroxypropyl methylcellulose, cellulose acetate, and combinations thereof; or
wherein the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof; or
wherein the pore-forming agent is selected from the group consisting of polyethylene glycol, hydroxypropyl methylcellulose, copovidone, povidone, and combinations thereof; or
wherein the anti-sticking agent is selected from the group consisting of talc powder, magnesium oxide, silicon dioxide, magnesium stearate, and combinations thereof; or
wherein the pH-independent sustained-release material is selected from the group consisting of methacrylate copolymer, ethyl cellulose, hydroxypropyl methylcellulose, cellulose acetate, and combinations thereof; and/or
wherein the pH-dependent coating film-forming material is selected from the group consisting of polymethacrylate, hydroxypropyl methylcellulose phthalate, polyacrylic acid resin, hydroxypropyl methylcellulose acetate succinate, and combinations thereof.

13. The composition according to claim 6,
wherein the sustained-release coating film-forming material is selected from the group consisting of methacrylate copolymer, ethyl cellulose, hydroxypropyl methylcellulose, cellulose acetate, and combinations thereof; or
wherein the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof; or
wherein the pore-forming agent is selected from the group consisting of polyethylene glycol, hydroxypropyl methylcellulose, copovidone, povidone, and combinations thereof; or
wherein the anti-sticking agent is selected from the group consisting of talc powder, magnesium oxide, silicon dioxide, magnesium stearate, and combinations thereof; or
wherein the pH-independent sustained-release material is selected from the group consisting of methacrylate copolymer, ethyl cellulose, hydroxypropyl methylcellulose, cellulose acetate, and combinations thereof; and/or
wherein the permeation control material is selected from glyceryl behenate and/or ethyl cellulose; or
wherein the permeation regulating material is selected from the group consisting of polyethylene glycol, hydroxypropyl methylcellulose, copovidone, povidone, polyoxyethylene ether glycerol butanediol ester, methyl silicone oil, zinc stearate, calcium stearate, aluminum stearate, sodium stearate, polysorbate, talc powder, silicon dioxide, and combinations thereof.

14. The composition according to claim 7,
wherein the pH-dependent coating film-forming material is selected from the group resin, hydroxypropyl methylcellulose acetate succinate, and combinations thereof; or
wherein the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof; or
wherein the anti-sticking agent is selected from the group consisting of talc powder, magnesium oxide, silicon dioxide, magnesium stearate, and combinations thereof; and/or
wherein the permeation control material is selected from glyceryl behenate and/or ethyl cellulose; or
wherein the permeation regulating material is selected from the group consisting of polyethylene glycol, hydroxypropyl methylcellulose, copovidone, povidone, polyoxyethylene ether glycerol butanediol ester, methyl silicone oil, zinc stearate, calcium stearate, aluminum stearate, sodium stearate, polysorbate, talc powder, silicon dioxide, and combinations thereof.

15. The composition according to claim 8,
wherein the sustained-release coating film-forming material is selected from the group consisting of methacrylate copolymer, ethyl cellulose, hydroxypropyl methylcellulose, cellulose acetate, and combinations thereof; or
wherein the plasticizer is selected from the group consisting of polyethylene glycol, glycerol, propylene glycol, diethyl phthalate or dibutyl phthalate, dibutyl sebacate, triethyl citrate or tributyl citrate, tributyl O-acetylcitrate, castor oil, silicone oil, glycerol triacetate, and combinations thereof; or
wherein the pore-forming agent is selected from the group consisting of polyethylene glycol, hydroxypropyl methylcellulose, copovidone, povidone, and combinations thereof; or
wherein the anti-sticking agent is selected from the group consisting of talc powder, magnesium oxide, silicon dioxide, magnesium stearate, and combinations thereof; or
wherein the pH-independent sustained-release material is selected from the group consisting of methacrylate copolymer, ethyl cellulose, hydroxypropyl methylcellulose, cellulose acetate, and combinations thereof;
wherein the pH-dependent coating film-forming material is selected from the group resin, hydroxypropyl methylcellulose acetate succinate, and combinations thereof; and
wherein the permeation control material is selected from glyceryl behenate and/or ethyl cellulose; or
wherein the permeation regulating material is selected from the group consisting of polyethylene glycol, hydroxypropyl methylcellulose, copovidone, povidone, polyoxyethylene ether glycerol butanediol ester, methyl silicone oil, zinc stearate, calcium stearate, aluminum stearate, sodium stearate, polysorbate, talc powder, silicon dioxide, and combinations thereof.

16. The composition according to claim 1, wherein the pH-independent sustained-release layer comprises 50% to 80% by weigth of a sustained-release coating film-forming material, 5% to 30% by weigth of a plasticizer, 10% to 35% by weigth of a pore-forming agent, and 0% to 10% by weigth of an anti-sticking agent, based on a total amount of the sustained-release coating film-forming material, the plasticizer, the pore-forming agent, and the anti-sticking agent; the pH-dependent coating layer comprises 50% to 90% by weigth of a pH-dependent coating film-forming material, 10% to 40% by weigth of a plasticizer, and 0 to 10% by weigth of an anti-sticking agent; and the time-dependent pulsatile controlled-release layer comprises 10% to 90% by weight of a permeation control material and 10% to 90% by weight of a permeation regulating material.

17. The composition according to claim 8, wherein the pH-independent sustained-release layer comprises 50% to 80% by weigth of a sustained-release coating film-forming material, 5% to 30% by weigth of a plasticizer, 10% to 35% by weigth of a pore-forming agent, and 0% to 10% by weigth of an anti-sticking agent, based on a total amount of the sustained-release coating film-forming material, the plasticizer, the pore-forming agent, and the anti-sticking agent; the pH-dependent coating layer comprises 50% to 90% by weigth of a pH-dependent coating film-forming material, 10% to 40% by weigth of a plasticizer, and 0 to 10% by weigth of an anti-sticking agent; and the time-dependent pulsatile controlled-release layer comprises 10% to 90% by weight of a permeation control material and 10% to 90% by weight of a permeation regulating material.

18. The composition according to claim 15, wherein the pH-independent sustained-release layer comprises 50% to 80% by weigth of the sustained-release coating film-forming material, 5% to 30% by weigth of the plasticizer, 10% to 35% by weigth of the pore-forming agent, and 0% to 10% by weigth of the anti-sticking agent, based on a total amount of the sustained-release coating film-forming material, the plasticizer, the pore-forming agent, and the anti-sticking agent; the pH-dependent coating layer comprises 50% to 90% by weigth of the pH-dependent coating film-forming material, 10% to 40% by weigth of the plasticizer, and 0 to 10% by weigth of the anti-sticking agent; and the time-dependent pulsatile controlled-release layer comprises 10% to 90% by weight of the permeation control material and 10% to 90% by weight of the permeation regulating material.

19. A method for preparing an intestine-targeted drug delivery composition, comprising the steps of:
(1) preparation of a pH-independent sustained-release layer
adding an active ingredient, a sustained-release coating film-forming material, and one or more pharmaceutically acceptable excipients selected from the group consisting of a plasticizer, a pore-forming agent, and an anti-sticking agent to an aqueous solution of an alcohol to form a sustained-release coating solution, and then coating blank microsphere cores with the sustained-release coating solution, for example, with a coating weight increase by 20 wt % to 100 wt %, to obtain sustained-release microspheres; or
adding a sustained-release coating film-forming material and one or more pharmaceutically acceptable excipients selected from the group consisting of a plasticizer, a pore-forming agent, and an anti-sticking agent to an aqueous solution of an alcohol to form a sustained-release coating solution; granulating an active ingredient, a binder, and a filler to obtain drug-containing microsphere cores, for example those with a particle size of 0.1 mm to 2 mm, 0.2 mm to 1 mm, or 0.2 mm to 0.6 mm; and then coating the drug-containing microsphere cores with the sustained-release coating solution, for example, with a coating weight increase by 20 wt % to 100 wt %, to obtain sustained-release microspheres; or
adding a sustained-release coating film-forming material and one or more pharmaceutically acceptable excipients selected from the group consisting of a plasticizer, a pore-forming agent, and an anti-sticking agent to an aqueous solution of an alcohol to form a sustained-release coating solution; coating blank microsphere cores with a solution containing an active ingredient and a binder to obtain drug-containing microsphere cores; and then coating the drug-containing microsphere cores with the sustained-release coating solution, for example, with a coating weight increase by 20 wt % to 100 wt %, to obtain sustained-release microspheres; or granulating an active ingredient, a binder, a filler, and a pH-independent sustained-release material to obtain sustained-release microspheres containing the active ingredient and the pH-independent sustained-release material;
(2) preparation of a pH-dependent coating layer
adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the sustained-release microspheres obtained in step (1) with the pH-dependent coating solution, for example, with a coating weight increase by 5 wt % to 50 wt %, to obtain pH-dependent microspheres; or
adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the sustained-release microspheres obtained in step (1) with the pH-dependent coating solution, for example, with a coating weight increase by 5 wt % to 50 wt %, to obtain pH-dependent microspheres; and mixing the pH-dependent microspheres with a filler, a disintegrant, and/or a lubricant, and then tableting to obtain a pH-dependent tablet, for example a pH-dependent tablet with a hardness of 30 to 100 N; or
mixing the sustained-release microspheres obtained in step (1) with a filler, a disintegrant, and/or a lubricant, and then tableting to obtain a compressed tablet, for example a compressed tablet with a hardness of 30 to 100 N; and adding a pH-dependent coating film-forming material and one or more pharmaceutically acceptable excipients selected from the group consisting of a plasticizer and an anti-sticking agent to an aqueous solution of an alcohol to form a pH-dependent coating solution, and then coating the compressed tablet with the pH-dependent coating solution, for example, with a coating weight increase by 5 wt % to 50 wt %, to obtain a pH-dependent tablet; and
(3) preparation of a time-dependent pulsatile controlled-release layer
adding a permeation control material and a permeation regulating material to an aqueous solution of an alcohol to form a time-dependent coating solution, and then coating the pH-dependent microspheres or the pH-dependent tablet with the time-dependent coating solution, for example, with a coating weight increase by 50 wt % to 400 wt %, to obtain the intestine-targeted drug delivery composition in microsphere form or tablet form.

20. A method for treating intestinal diseases, such as inflammatory bowel diseases including Crohn's disease and ulcerative colitis, in a patient in need thereof, comprising administering the intestine-targeted drug delivery composition according to claim 1 to the patient.
